(19) United States
(12) Patent Application Publication
(10) Pub. No.: US 2025/0009323 A1
(43) Pub. Date: Jan. 9, 2025

(54) MEDICAL IMAGING SYSTEM, IMAGING CONTROL APPARATUS, MEDICAL IMAGING METHOD, AND RECORDING MEDIUM

(71) Applicant: Konica Minolta, Inc., Tokyo (JP)

(72) Inventor: Ryohei ITO, Tokyo (JP)

(21) Appl. No.: 18/762,092

(22) Filed: Jul. 2, 2024

(30) Foreign Application Priority Data
Jul. 7, 2023 (JP) 2023-111929

(51) Int. Cl. A61B 6/00

(52) U.S. Cl. CPC A61B 6/547

(57) ABSTRACT

A medical imaging system includes a hardware processor that acquires imaging order information including information on positioning, determines whether the acquired imaging order information satisfies predetermined conditions, and outputs imaging support information before start of the positioning when the imaging order information satisfies the predetermined conditions. In one embodiment, the imaging order information includes information on an imaging direction of an imaging site.

### CROSS-REFERENCE TO RELATED APPLICATION

The disclosure of Japanese Patent Application No. 2023-111929 filed on Jul. 7, 2023, including description, claims, drawings, and abstract, is incorporated herein by reference in its entirety.

### BACKGROUND OF THE INVENTION

### Technical Field

The present invention relates to a medical imaging system, an imaging control apparatus, a medical imaging method, and a recording medium.

### Description of Related Art

In the medical field, diagnosis based on radiographs obtained through radiographing has been conventionally performed. In the radiographing, positioning of a patient is performed on the basis of an imaging direction and the like of the input order information. In order to succeed in radiographing, it is important to appropriately perform the positioning of a patient.

Here, the radiographing is imaging in which a three dimensional subject image is projected onto a two dimensional plane. A distance between a subject and a detector in the radiographing varies depending on the position of the subject. Therefore, in some cases, a part of the subject may be imaged in an enlarged manner depending on the imaging direction. Consequently, the imaging direction at the time of radiographing is basically determined for each imaging portion in many cases.

International Publication No. 2019-208006 discloses a radiography apparatus that determines an orientation of a subject. The radiography apparatus determines the orientation of the subject on the basis of the visible image of the subject acquired by an image acquisition device. Next, when determining that the orientation of the subject is different from a protocol, the radiography apparatus issues a warning and prohibits radiographing for the subject.

### SUMMARY OF THE INVENTION

However, in the conventional technology, after the positioning of the patient is completed, it is determined whether the positioning of the subject is correct with respect to the protocol. Therefore, when it is determined to change the orientation of the patient, significant time is wasted and a burden on the patient increases. In particular, in the case of radiographing that is not performed in a general imaging direction, a radiologist may erroneously perform the positioning of a patient in the general imaging direction.

Thus, it is an object of the present invention to provide a medical imaging system, an imaging control apparatus, a medical imaging method, and a program that are capable of efficiently performing positioning and reducing a burden on a patient in radiographing.

To achieve at least one of the abovementioned objects, according to an aspect of the present invention, a medical imaging system reflecting one aspect of the present invention includes:
a hardware processor that acquires imaging order information including information on positioning,
determines whether the acquired imaging order information satisfies predetermined conditions, and
outputs imaging support information before start of the positioning when the imaging order information satisfies the predetermined conditions.

To achieve at least one of the abovementioned objects, according to an aspect of the present invention, an imaging control apparatus reflecting one aspect of the present invention includes:
a hardware processor that acquires imaging order information including information on positioning,
determines whether the acquired imaging order information satisfies predetermined conditions, and
outputs imaging support information before start of the positioning when the imaging order information satisfies the predetermined conditions.

To achieve at least one of the abovementioned objects, according to an aspect of the present invention, a medical imaging method reflecting one aspect of the present invention causes a hardware processor to perform:
acquiring imaging order information including information on positioning;
determining whether the acquired imaging order information satisfies predetermined conditions; and
outputting imaging support information before start of the positioning when the imaging order information satisfies the predetermined conditions.

To achieve at least one of the abovementioned objects, according to an aspect of the present invention, a recording medium reflecting one aspect of the present invention stores a program that causes a computer to perform:
acquiring imaging order information including information on positioning;
determining whether the acquired imaging order information satisfies predetermined conditions; and
outputting imaging support information before start of the positioning when the imaging order information satisfies the predetermined conditions.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 is a diagram illustrating a schematic configuration of a medical imaging system according to the present embodiment;

FIG. 2 is a block diagram of an imaging control apparatus according to the present embodiment;

FIG. 3 is a diagram illustrating a list of representative imaging sites, imaging directions that can be specified for the imaging site, and a normal imaging direction and reasons for the normal imaging direction according to the present embodiment;

FIG. 4 is a diagram illustrating an example of a setting screen for setting notification necessity information indicating whether to provide notification of imaging support information according to an imaging site and an imaging direction, and the like according to the present embodiment;

FIG. 5 is a diagram illustrating an example of a configuration of a table stored in the storage according to the present embodiment;

FIG. 6 is a flowchart illustrating an example of an operation of the imaging control apparatus at the time of imaging a radiograph of a subject according to the present embodiment;

FIG. 7 is a diagram illustrating an example of a configuration of an imaging screen displayed on a display part of the imaging control apparatus according to the present embodiment;

FIG. 8 is a diagram illustrating an example of imaging support information displayed on the imaging screen of the display part according to the present embodiment;

FIG. 9 is a diagram illustrating an example of a configuration of an imaging screen displayed on a display part when an imaging site included in order information according to Other Embodiment 1 is an imaging site with very low frequency;

FIG. 10 is a diagram illustrating an example of imaging support information displayed in an examination list of the display part according to Other Embodiment 1;

FIG. 11 is a diagram illustrating an example of an imaging screen displayed on a display part at the time of double focus imaging according to Other Embodiment 2; and

FIG. 12 is a diagram illustrating a schematic configuration of a medical imaging system according to Other Embodiment 3.

### DETAILED DESCRIPTION

Hereinafter, one or more embodiments of the present invention will be described with reference to the drawings. However, the scope of the invention is not limited to the disclosed embodiments.

### [Configuration Example of Medical Imaging System 10A]

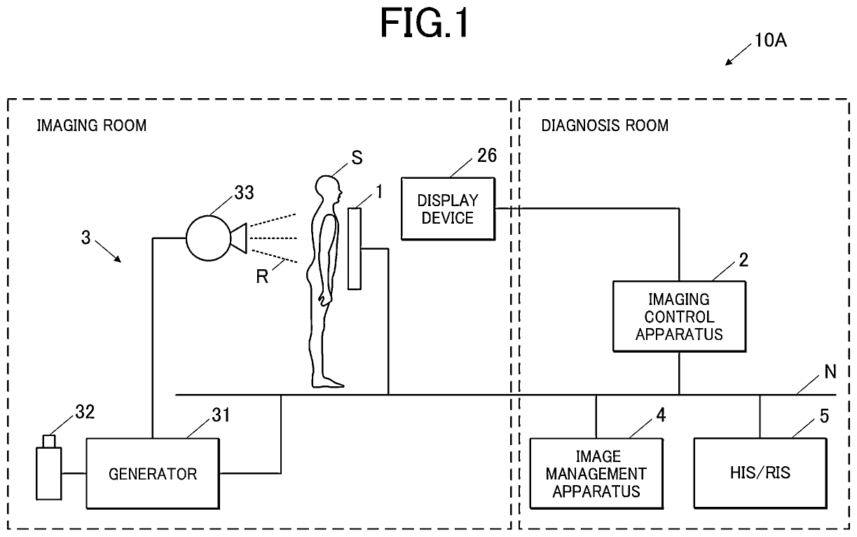

FIG. 1 is a diagram illustrating a schematic configuration of a medical imaging system 10A. The medical imaging system 10A includes a radiographic imaging apparatus 1, an imaging control apparatus 2, a radiation generation apparatus 3, an image management apparatus 4, and a HIS/RIS 5. Hereinafter, the radiographic imaging apparatus may be referred to as the imaging apparatus 1, and the radiation generation apparatus may be referred to as the generation apparatus 3. HIS is an abbreviation for Hospital Information System and is a hospital information system. RIS is an abbreviation for Radiology Information System and is a radiology department information system. In the present embodiment, the imaging apparatus 1 and the generation apparatus 3 are arranged in an imaging room. The imaging control apparatus 2, the image management apparatus 4, and the HIS/RIS 5 are arranged in a diagnosis room.

The imaging apparatus 1, the imaging control apparatus 2, the generation apparatus 3, and the image manager 4 are communicably connected to each other through a network N. Examples of the network N include a LAN, a WAN, or the Internet. LAN is an abbreviation for Local Area Network. WAN is an abbreviation for Wide Area Network. A communication system of the network N may be wired or wireless.

The generation apparatus 3 includes a generator 31, a switch 32, and a radiation source 33.

The generator 31 applies, to the radiation source 33 including, for example, a tube, a voltage according to preset imaging conditions, in response to operation of the switch 32. The generator 31 may include an operation part that receives input of radiation irradiation conditions and the like.

When the generator 31 applies a voltage to the radiation source 33, the radiation source 33 emits radiation R having a dose according to the applied voltage. The radiation R is, for example, X-rays.

The generation apparatus 3 generates the radiation R in a manner according to the type of radiograph, for example, a still image or a dynamic image. To be specific, in generating a still image, the generation apparatus 3 emits the radiation R only once in response to the switch 32 being pressed once. In generating a dynamic image, for example, in response to the switch 32 being pressed once, the generation apparatus 3 repeats emission of pulsed radiation R multiple times per predetermined period of time.

The imaging apparatus 1 produces digital image data including an imaging site of a subject. For the imaging apparatus 1, for example, a portable FPD is used. FPD is an abbreviation for Flat Panel Detector.

Although not illustrated, the imaging device 1 includes, for example, an imaging element, a sensor substrate, a scanner, a reader, a controller, and a communicator. The imaging element generates electric charges according to the dose of received radiation R. In the sensor substrate, switch elements are arranged two-dimensionally (in a matrix), and in a two dimensional array (matrix), and accumulate and discharge the electric charges. The scanner switches on and off of each switch element. The reader reads, as a signal value, an amount of electric charge emitted from each pixel. The controller generates a radiograph from a plurality of signal values read by the reader. The communicator transmits data of the generated radiograph, various signals and the like to another apparatus such as the imaging control apparatus 2, and receives various kinds of information and various signals from another apparatus.

The imaging apparatus 1 accumulates and discharges electric charges and reads signal values in synchronization with the timing at which the radiation R is emitted from the generation apparatus 3. The imaging apparatus 1 generates image data of a still image or image data of a dynamic image on the basis of the read signal values and the like. The imaging apparatus 1 outputs the generated image data to the imaging control apparatus 2. The imaging apparatus 1 may be integrated with the generation apparatus 3.

The imaging control apparatus 2 sets imaging conditions for the imaging apparatus 1, the generation apparatus 3, and the like, and controls a reading operation of the radiograph imaged by the imaging apparatus 1. The imaging control apparatus 2 is also referred to as a console, and is constituted by, for example, a personal computer. When an imaging direction of order information (imaging order information) is different from a normal imaging direction, the imaging control apparatus 2 provides notification of imaging support information 9a before the start of positioning of the patient. That is, when the imaging direction of the order information is an imaging direction with low imaging frequency, the imaging control apparatus 2 provides notification of the imaging support information 9a before the start of positioning of the patient. Here, the imaging support information 9a is information for assisting a user such as a radiologist in positioning a patient at the time of imaging. To be specific, the imaging support information 9a is information for alerting the user that the imaging direction of the order information is different from the normal imaging direction.

The imaging conditions include, for example, patient conditions related to a subject S, irradiation conditions related to the irradiation of the radiation R, and image reading conditions related to the image reading of the imaging apparatus 1. The patient conditions include, for example, an imaging site, an imaging direction, and a body type. The irradiation conditions include, for example, tube voltage (kV), tube current (mA), irradiation time (ms), and current time product (mAs value). The image reading conditions include, for example, an image size, and a frame rate. The imaging control apparatus 2 may automatically set the imaging conditions on the basis of the order information acquired from the HIS/RIS 5 or the like. Alternatively, the imaging control apparatus 2 may manually set the imaging conditions in response to operations made by a user such as a radiologist on the operation part 21, for example.

A display device 26 is connected to the imaging control apparatus 2. The display device 26 is connected to the imaging control apparatus 2 using an interface such as HDMI (registered trademark), for example. HDMI is an abbreviation for High Definition Multimedia Interface. The communication method may be wired or wireless. The display device 26 is arranged in the imaging room so that a user or the like in the imaging room can visually confirm the display device 26. The display device 26 includes, for example, a general-purpose monitor such as a liquid crystal display, which will be described below. For example, the same image as the image displayed on a display part 22 of the imaging control apparatus 2 can be displayed on a screen of the display device 26. To be specific, on the screen of the display device 26, in addition to an imaging screen, the imaging support information 9a for alerting the user when the imaging direction of the order information is different from the normal imaging direction is displayed. This enables the user in the imaging room to also confirm that the imaging direction specified by the current order information is a rare imaging direction with low frequency of use. In the present embodiment, the display device 26 functions as an external device and an imaging apparatus that display the imaging support information 9a output from the imaging control apparatus 2. Note that depending on the equipment, space, or the like of the facility, the medical imaging system 10A may have a structure in which the display device 26 is not arranged.

The image management apparatus 4 manages the image data generated by the imaging apparatus 1. The image management apparatus 4 is a picture archiving and communication system, a diagnostic imaging workstation, or the like. The picture archiving and communication system may be referred to as PACS. PACS is an abbreviation for Picture Archiving and Communication System.

The HIS/RIS 5 receives, for example, the order information on the radiographing of the patient from a doctor or the like, and transmits the received order information to the imaging control apparatus 2. The order information includes, for example, various kinds of information such as an ID, an imaging site, an imaging direction, and a body type of the patient.

### [Block Diagram of Imaging Control Apparatus 2]

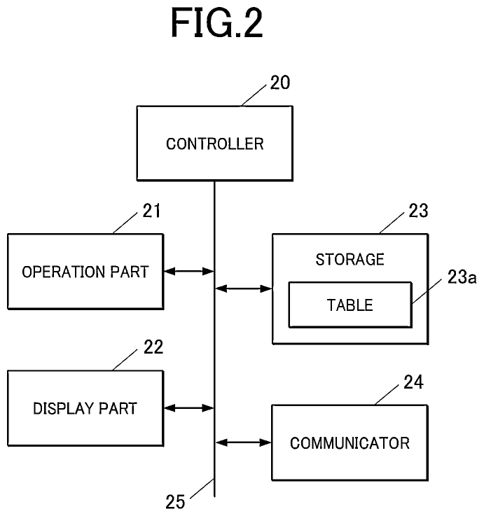

FIG. 2 is a block diagram of the imaging control apparatus 2. The imaging control apparatus 2 includes a controller 20 (i.e., a hardware processor), an operation part 21, a display part 22, a storage 23, and a communicator 24. The controller 20, the operation part 21, the display part 22, the storage 23, and the communicator 24 are communicably connected via, for example, a bus 25.

The controller 20 includes at least a processor such as a CPU and a memory such as a RAM. The CPU loads various programs stored in the storage 23 into the RAM, and performs various functions by executing the programs. CPU is an abbreviation for Central Processing Unit. RAM is an abbreviation for Random Access Memory. Note that the CPU may be constituted by a single processor or a plurality of processors.

In the present embodiment, the controller 20 functions as an acquirer, a determiner, a setter, a specifier, and a notifier. Each function of the acquirer, the determiner, the setter, the specifier, and the notifier is performed by the processor of the controller 20 executing a program stored in the storage 23 or the like.

The acquirer acquires imaging order information including information on positioning of a patient. Examples of the information on the positioning include an imaging site and an imaging direction included in the order information. The information on the imaging direction is an irradiation direction of the radiation R in a predetermined imaging direction. To be specific, when one side of the subject S comes into contact with the imaging apparatus 1, a direction from the other side of the subject S toward the one side in the imaging direction coincides with the irradiation direction of the radiation R.

The determiner determines whether the acquired imaging order information satisfies the predetermined conditions.

The setter can set, as the predetermined conditions, information on an imaging direction and notification necessity information as to whether to provide notification of the imaging support information 9a in association with each other. The information on the imaging direction and the notification necessity information as to whether to provide notification of the imaging support information 9a are stored in the storage 23 as a table 23a.

The specifier specifies the notification necessity information associated with the information on the imaging direction stored in the table 23a on the basis of the information on the imaging direction of the acquired imaging order information.

The determiner determines that the predetermined conditions are satisfied when the specified notification necessity information is information indicating that notification of the imaging support information 9a is to be provided (ON).

When the imaging order information satisfies the predetermined conditions, the notifier provides the notification of the imaging support information 9a before the start of positioning.

The operation part 21 receives an instruction according to various input operations from a user, converts the received instruction into an operation signal, and outputs the operation signal to the controller 20. The operation part 21 includes, for example, at least one of a mouse, a keyboard, a switch, a button and the like. The operation part 21 may be, for example, a touch screen integrally combined with a display. The operation part 21 may be, for example, a user interface such as a microphone that receives a voice input. The operation part 21 of the present embodiment receives setting of the notification necessity information of the imaging support information 9a according to the imaging direction and the like, the imaging support information 9a associated with the notification necessity information, and the like.

The display part 22 displays a radiograph based on the image data received from the imaging apparatus 1, a GUI for receiving various input operations from the user, and the like. GUI is an abbreviation for Graphical User Interface. The display part 22 is, for example, a display such as a liquid crystal display or an organic EL display. EL is an abbreviation for Electro Luminescence. The display part 22 of the present embodiment displays, on the imaging screen, the imaging support information 9a for alerting the user when the imaging direction of the order information is different from the normal imaging direction, or the like.

The storage 23 stores, for example, a system program, an application program, and various kinds of data. The storage 23 includes, for example, at least one storage module among an HDD, an SSD, a ROM, a RAM, and the like. HDD is an abbreviation for Hard Disk Drive. SSD is an abbreviation for Solid State Drive. ROM is an abbreviation for Read Only Memory. The storage 23 of the present embodiment stores the table 23a in which the imaging direction is associated with the notification necessity information indicating whether to notify the user of the imaging support information 9a according to the imaging direction.

The communicator 24 is, for example, a communication module including an NIC, a receiver, a transmitter, and the like. NIC is an abbreviation for Network Interface Card. The communicator 24 communicates various types of data such as image data among the imaging apparatus 1, the image management apparatus 4, and the like through the network N.

Note that the imaging control apparatus 2 may be configured not to include the operation part 21 and the display part 22. In this case, the imaging control apparatus 2 may receive a control signal from an operation part provided in an external device connected via the communicator 24. The imaging control apparatus 2 may output an image signal to a display part provided in the external device to display a radiograph or the like. The external device may be the image management apparatus 4 or the like, or may be another device.

### [General Imaging Direction of Representative Imaging Site]

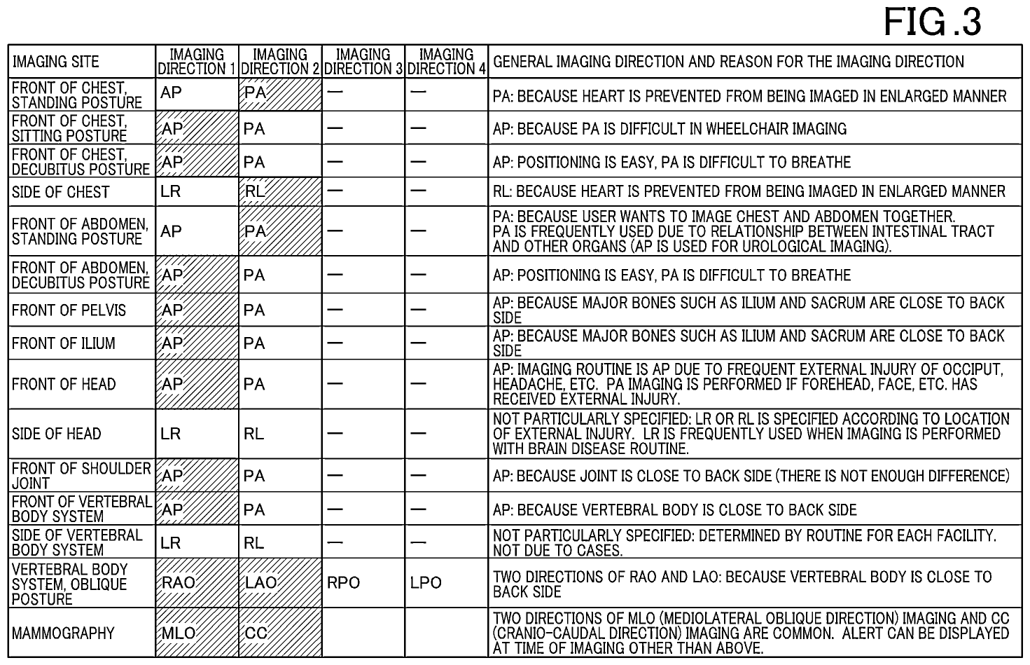

Next, a general imaging direction in a representative imaging site will be described. FIG. 3 illustrates a list of a representative imaging site, imaging directions that can be specified for the imaging site, and a normal imaging direction and reasons for the normal imaging direction.

First, terms of imaging directions used in the drawings will be described.

AP is an abbreviation for Anterior-Posterior. The AP imaging means that imaging is performed in a state in which the back side of the body of the patient is in contact with the imaging apparatus 1 such that the radiation passes through the patient from the front side to the back side.

PA is an abbreviation for Posterior-Anterior. The PA imaging means that imaging is performed in a state in which the abdomen side of the body of the patient is in contact with the imaging apparatus 1 such that the radiation passes through the patient from the back side to the front side.

LR is an abbreviation for Left-Right. The LR imaging means that imaging is performed in a state in which the right side of the body of the patient is in contact with the imaging apparatus 1 such that the radiation passes through the patient from the left side to the right side.

RL is an abbreviation for Right-Left. The RL imaging means that imaging is performed in a state in which the left side of the body of the patient is in contact with the imaging apparatus 1 such that radiation passes through the patient from the right side to the left side.

The routine refers to general imaging at the site.

As illustrated in FIG. 3, in a case where the imaging site is “front of chest, standing posture”, two directions of “AP” and “PA” can be specified as the imaging directions. However, when the imaging site is “front of chest, standing posture”, “PA” is generally specified as the imaging direction. The reason why “PA” is specified is because the heart is prevented from being imaged in an enlarged manner. Furthermore, it has been confirmed that “PA” is overwhelmingly specified as the imaging direction also from the investigation results of the specification rate of PA and AP for “front of chest, standing posture” in a plurality of hospitals.

Also for other imaging sites, the imaging direction to be specified is usually determined in order to accurately image radiographs and to reduce a burden at the time of positioning. For example, when the imaging site is “front of chest, sitting posture”, “AP” is specified as the imaging direction. The reason why “AP” is specified is because, for example, in the case of imaging of a wheelchair patient, the imaging in “PA” is difficult.

However, an imaging direction with low frequency of use may be rarely specified depending on the disease state of the patient. For example, this is a case where “AP” is specified as the imaging direction in a case where the imaging site is “front of chest, standing posture”. In such a case, a user such as a radiologist in charge may overlook the imaging direction ordered by a doctor, resulting in imaging in an imaging direction different from the order. This is because the user believes, from the input imaging direction, that the imaging direction of the order is the normal imaging direction “PA” with high imaging frequency. In particular, the user tends to erroneously confirm the imaging direction under such circumstances that the user is busy.

Then, in the present embodiment, in a case where an order in which a specific imaging direction different from the normal imaging direction is specified is input, in order to alert the user that the specific imaging direction is different from the normal imaging direction, the notification to that effect is provided before the start of positioning of the patient. In a case where the imaging site is “head, side” or the like, each of the two directions of “LR” and “RL” is generally specified, and there are cases where the routine imaging is not determined. In the present embodiment, such imaging sites may be controlled without using the imaging support information 9a for alerting the user. Furthermore, control may be performed to notify the user of the imaging support information 9a in a case where any one of the two directions is specified.

### [Configuration Example of Setting Screen 70]

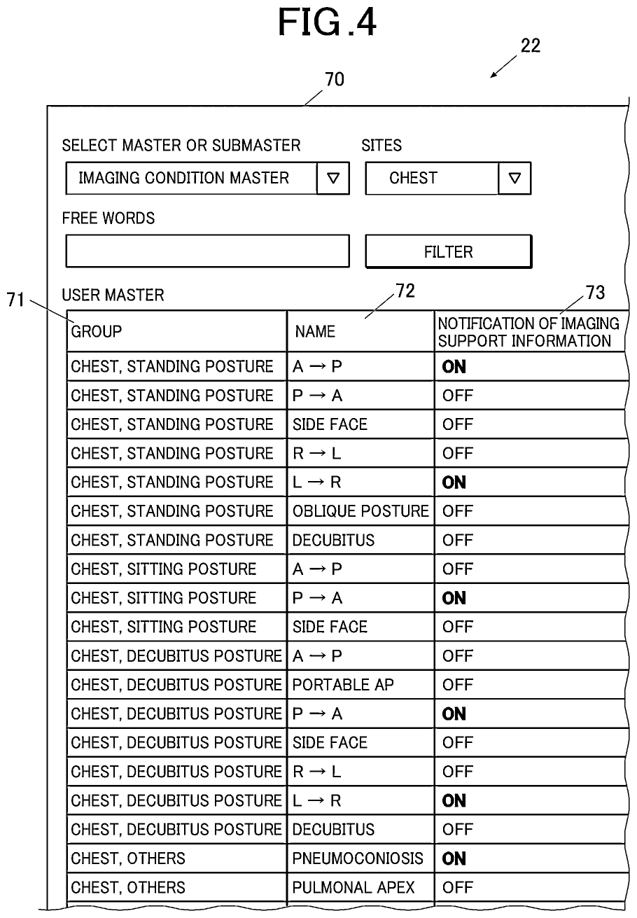

FIG. 4 illustrates an example of a setting screen 70 for setting notification necessity information indicating whether to provide notification of the imaging support information 9a according to an imaging site and an imaging direction, and the like. The controller 20 causes the display part 22 to display the setting screen 70 on which the information on the imaging direction and the notification necessity information as to whether to provide notification of the imaging support information 9a can be set in association with each other. The setting screen 70 may be displayed, for example, in response to a user's selection of a setting button or the like displayed on the display part 22.

The setting screen 70 is provided with a site setter 71, a direction setter 72, and a notification necessity setter 73. The site setter 71 has an input field for inputting an imaging site. The direction setter 72 has an input field for inputting a imaging direction. The notification necessity setter 73 has an input field for inputting ON or OFF of notification necessity information as to whether to provide notification of the imaging support information 9a.

To be specific, when “chest, standing posture” is input in the site setter 71 and “AP” is input in the direction setter 72, the user inputs “ON” as the notification of the imaging support information 9a. This is because in “chest, standing posture”, “AP” is a direction with low imaging frequency and is different from the normal imaging direction, and thus it is necessary to alert the user to the fact. On the other hand, when “chest, standing posture” is input to the site setter 71 and “PA” is input to the direction setter 72, the user inputs “OFF” as the notification of the imaging support information 9a. This is because in “chest, standing posture”, “PA” has high frequency of use and is used for routine imaging, and thus it is not necessary to alert the user.

All patterns of combinations of assumed imaging sites and imaging directions may be input in advance to the site setter 71 and the direction setter 72. In this case, the user inputs the notification of the imaging support information 9a associated with the imaging sites and the imaging directions from the notification necessity setter 73. Note that the notification (ON or OFF) of the imaging support information 9a may be input in advance to the notification necessity setter 73 by using statistics or the like of the imaging frequency of the imaging direction. For example, in the case of an imaging direction with high imaging frequency, OFF is set in the notification necessity setter 73 in advance. In the case of an imaging direction with a low imaging frequency, ON is set in the notification necessity setter 73 in advance. At least one of the imaging site, the imaging direction, and the notification of the imaging support information 9a may be a pull-down system in which the user selects a specific item from a plurality of items prepared in advance. Alternatively, the user may directly input character information such as the imaging direction.

[Configuration Example of Table 23a]

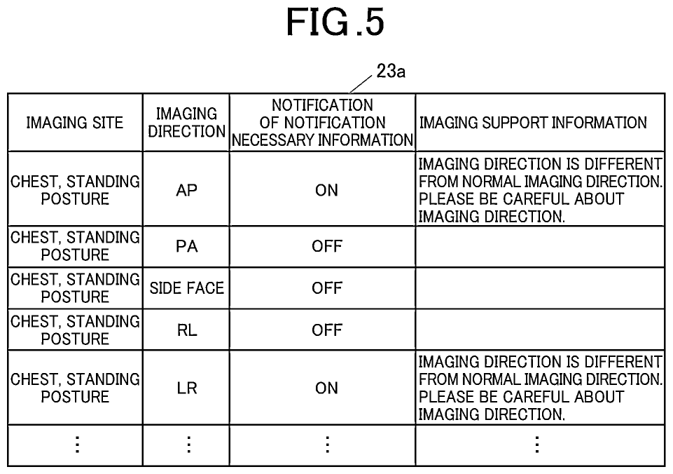

Next, the table 23a in which various kinds of information set on the setting screen 70 are reflected will be described. FIG. 5 illustrates an example of a configuration of the table 23a stored in the storage 23. In the table 23a, an imaging site, an imaging direction, notification of notification necessity information, and the imaging support information 9a are associated with each other. The imaging site, the imaging direction, the notification of the notification necessity information, and the imaging support information 9a in Table 23a can be configured by the input information set on the setting screen 70 described above. Note that in a case where the notification of the notification necessity information is ON, it may be possible to input or select a text that forms the imaging support information 9a in the setting screen 70. For example, in the setting screen 70, a plurality of fixed phrases forming the imaging support information 9a may be prepared in advance so that the user can select a specific text.

To be specific, in a case where the imaging site is “chest, standing posture”, the imaging direction is “AP”, and the notification of the notification necessity information is “ON”, the following imaging support information 9a is stored in association with them. The imaging support information 9a includes, for example, a text for alerting the user “The imaging direction is different from the normal imaging direction. Please be careful about the imaging direction.”. On the other hand, in a case where the imaging site is “chest, standing posture”, the imaging direction is “PA”, and the notification of the notification necessity information is “OFF”, the imaging support information 9a is not associated with them.

The setting of the notification of the imaging support information 9a may be performed using a machine learning model (learned model) without using the setting screen 70 and the table 23a. The machine learning model may be, for example, a regression model such as polynomial regression, multiple regression, support vector regression, or random forest regression. Alternatively, the machine learning model may be another model such as a neural network. The machine learning model is a model learned on the basis of certain training data. The training data is a data set in which information including an imaging direction different from a normal imaging direction of each imaging site is used as input data and imaging support information 9a to be output in the case of the imaging direction different from the normal imaging direction is used as correct output data. Thus, when an imaging direction different from the normal imaging direction in an imaging site is input, the machine learning model outputs the imaging support information 9a associated with the imaging direction as estimation data.

### [Operation Example of Imaging Control Apparatus 2]

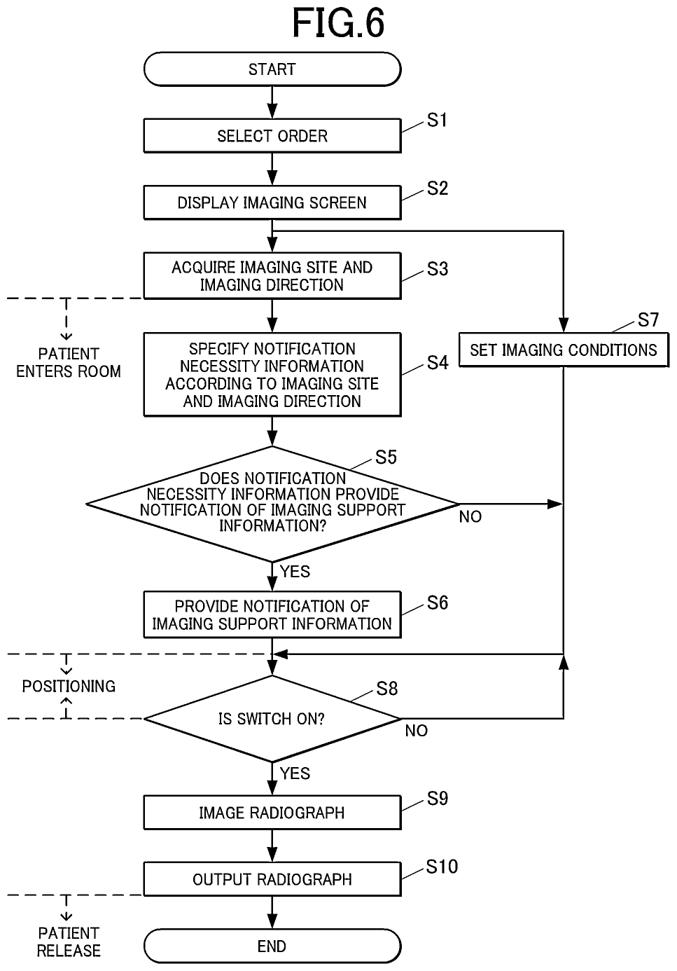

FIG. 6 is a flowchart illustrating an example of the operation of the imaging control apparatus 2 at the time of imaging a radiograph of the subject S.

The order information input by the doctor or the like is transmitted from the HIS/RIS 5 to the imaging control apparatus 2. The communicator 24 of the imaging control apparatus 2 receives the order information. The user selects predetermined order information from a examination list displayed on the screen of the display part 22 by operating the operation part 21. The controller 20 acquires the order information selected by the user (Step S1).

Upon acquiring the predetermined order information, the controller 20 causes the display part 22 to display an imaging screen 80 (Step S2).

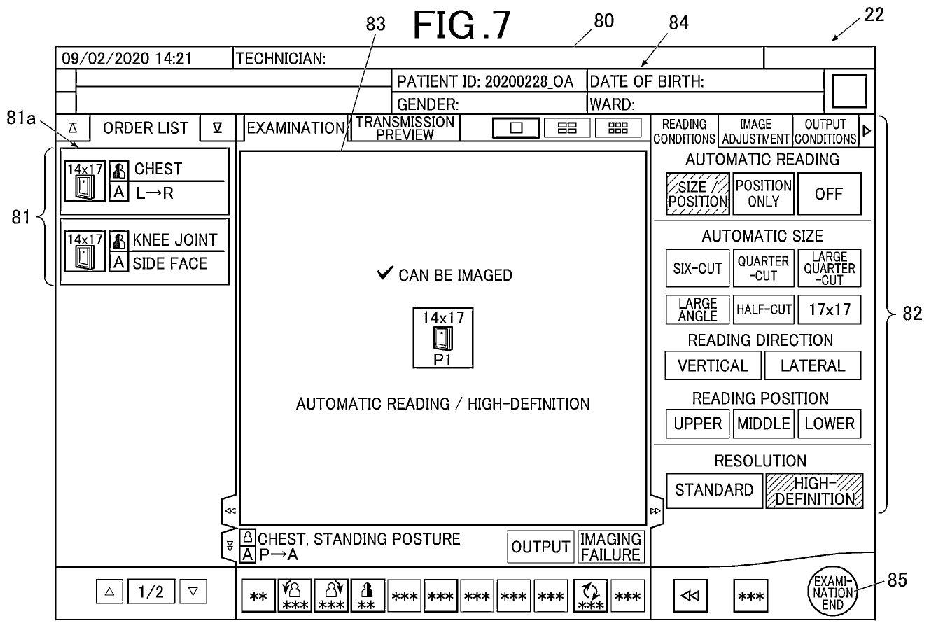

FIG. 7 illustrates an example of a configuration of the imaging screen 80 displayed on the display part 22 of the imaging control apparatus 2. The imaging screen 80 is provided with an imaging selector 81, a condition setter 82, an image display area 83, a patient information display part 84, and an output button 85. In the imaging selector 81, for example, the pieces of order information selected from the examination list are displayed. The order information includes, for example, imaging contents such as an imaging site and an imaging direction, and a thumbnail image indicating the imaged radiograph. Note that in FIG. 7, no thumbnail image is displayed in the imaging selector 81 because no radiograph has been imaged. The condition setter 82 is provided with, for example, a button for setting imaging conditions of a radiograph, a button for performing image adjustment of a imaged radiograph, and the like. In the image display area 83, a radiograph imaged by the imaging apparatus 1 is displayed on the basis of the set imaging conditions or the like.

When Step S2 is completed, the process branches into Step S3 and Step S6. In the present embodiment, the processes from Step S3 to Step S5 and the process of Step S6 are performed in parallel. Note that the processes from Step S3 to Step S6 may be serial processes performed in a sequential order.

First, the process of Step S3 will be described. The controller 20 acquires the imaging site and the imaging direction included in the order information selected by the imaging selector 81 (Step S3). The patient enters the imaging room by a call from a user who is, for example, a radiologist. At this stage, the positioning of the patient has not been started. Note that the timing at which the patient enters the room is not limited to the timing after the process of Step S3, and may be the timing before the process of Step S3 as long as the waiting time for the patient does not become long.

The controller 20 specifies the notification availability information indicating whether to provide notification of the imaging support information 9a on the basis of the acquired imaging site and imaging direction (Step S4). For example, the controller 20 refers to the table 23a and acquires the notification availability information indicating whether to provide the notification of the imaging support information 9a associated with the imaging site and the imaging direction.

The controller 20 determines whether the acquired notification availability information is ON (Step S5). That is, the controller 20 determines whether the predetermined conditions for displaying the imaging support information 9a are satisfied. If the acquired notification availability information is not ON, that is, if the controller 20 determines that the notification availability information is OFF, the controller 20 proceeds to Step S8. On the other hand, when determining that the notification of the acquired notification availability information is ON, the controller 20 proceeds to Step S7.

Next, Step S6 which is branched from Step S2 will be described. When predetermined order information is selected by the imaging selector 81 or the like, the controller 20 sets imaging conditions for each of the imaging apparatus 1 and the generation apparatus 3 (Step S6). The imaging conditions include image reading conditions to be set for the imaging apparatus 1 and irradiation conditions to be set for the generation apparatus 3. For example, the controller 20 sets the image reading conditions for the imaging apparatus 1 on the basis of the imaging site, the imaging direction, and the like of the selected order information. The controller 20 sets the irradiation conditions for the generation apparatus 3 on the basis of the imaging site, the imaging direction, and the like of the selected order information.

The imaging conditions may be manually set by the user. To be specific, the controller 20 may set the image reading conditions received by user's input operation on the condition setter 82 for the imaging apparatus 1. The controller 20 may set the radiation irradiation conditions received by user's input operation on the operation panel of the generation apparatus 3 for the generation apparatus 3. When Step S6 is completed, the controller 20 proceeds to Step S7. Note that the imaging conditions may be set after notification of the imaging support information 9a (which will be described later) and immediately before imaging depending on conditions such as the body type of the patient.

If the notification availability information is ON after the imaging conditions are set, the controller 20 provides notification of the imaging support information 9a (Step S7). For example, the controller 20 refers to the table 23a and acquires the imaging support information 9a associated with the imaging site, the imaging direction, and the notification availability information. The controller 20 causes the acquired imaging support information 9a to be displayed on the imaging screen 80 of the display part 22 before the start of positioning. In practice, the controller 20 can cause the imaging support information 9a to be displayed on the imaging screen 80 of the display part 22 immediately after the transition to the imaging screen 80. After confirming the imaging support information 9a on the imaging screen 80, the user starts positioning for the patient. Note that the imaging support information 9a may be displayed on the screen of the display device 26 in the imaging room in addition to the display part 22.

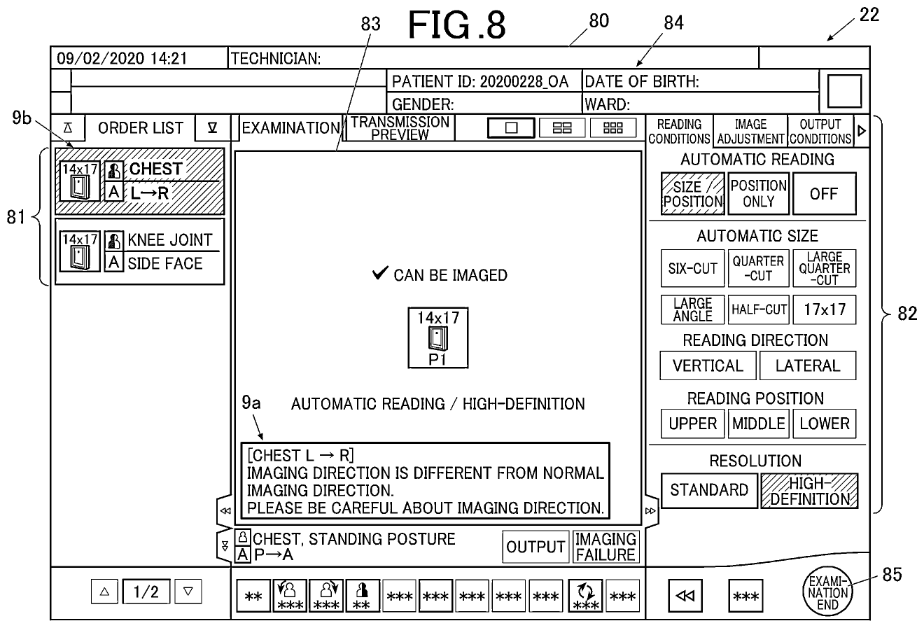

FIG. 8 illustrates an example of the imaging support information 9a displayed on the imaging screen 80 of the display part 22. When the notification availability information is ON, the controller 20 displays the imaging support information 9a on a part of the image display area 83. That is, when the imaging direction of the order information is different from the normal imaging direction, the controller 20 causes the display part 22 to display the imaging support information 9a before the start of positioning of the patient.

To be specific, when the order information in which the imaging site is “chest, standing posture” and the imaging direction is “LR” is input, the following imaging support information 9a is displayed on the imaging screen 80. The imaging support information 9a includes, for example, a text “The imaging direction is different from the normal imaging direction [chest L→R]. Please be careful about the imaging direction.”. This enables the user to confirm, before the start of positioning of the patient, that the imaging direction different from the normal imaging direction is input as the order information.

In FIG. 8, the imaging support information 9a is displayed on the lower left side of the imaging screen 80, but the display position is not limited to this. In addition to displaying the imaging support information 9a on the image display area 83, the display of the order information on the imaging selector 81 may be changed so as to function as imaging support information 9B. The imaging support information 9B is displayed such that, for example, the color and size of the characters “chest L→R” of the order information on the imaging selector 81 are different from those of the order information of the normal imaging. Note that the imaging support information 9B may be displayed in a font, brightness, or the like different from those of the order information of the normal imaging.

The imaging support information 9a may be other than the display by the display part 22. For example, the imaging control apparatus 2 may be provided with a speaker functioning as a notifier. The controller 20 may output, as the imaging support information 9a, sound effects or a voice indicating that the imaging direction is different from the normal imaging direction from the speaker. The notification of the imaging support information 9a may be performed by any one of the display part 22 and the speaker.

In the present embodiment, the positioning of the patient is performed after the display of the imaging support information 9a. By visually confirming the imaging support information 9a displayed on the imaging screen 80, the user can determine correct positioning according to the imaging direction of the order information. To be specific, when the order information in which the imaging site is “chest, standing posture” and the imaging direction is “LR” is input, the positioning is performed as follows. The user guides the patient such that the right side of the patient as the subject S comes into contact with the imaging apparatus 1 between the radiation source 33 of the generation apparatus 3 and the imaging apparatus 1. Note that if the imaging support information 9a is not displayed (Step S5: NO), the imaging direction is the normal imaging direction, and therefore, the positioning is performed in the normal manner.

The controller 20 determines whether the switch 32 has been turned on by the user (Step S8). When determining that the switch 32 has not been turned on by the user, the controller 20 returns to Step S8. In this case, the controller 20 waits until the switch 32 is turned on. On the other hand, if determining that the switch 32 has been turned on by the user, the controller 20 proceeds to Step S9. Note that in a case where the imaging support information 9a is displayed on the imaging screen 80, for example, a confirmation button for answering whether the user has confirmed the imaging support information 9a may be provided. In this case, when the confirmation button is selected by the user, the controller 20 may proceed to the next operation, for example, an operation of turning on the switch 32.

The controller 20 controls the imaging apparatus 1, the generation apparatus 3, and the like to image a radiograph of the subject S (Step S9). The generation apparatus 3 emits the radiation R to the imaging site of the subject S. The imaging apparatus 1 detects the radiation R transmitted through the subject S from the generation apparatus 3, and generates image data including an imaging site on the basis of the detected radiation R. The imaging apparatus 1 transmits the generated image data to the imaging control apparatus 2. The controller 20 of the imaging control apparatus 2 causes the radiograph based on the image data received from the imaging apparatus 1 to be displayed on the image display area 83 of the imaging screen 80.

The controller 20 adds patient information, examination information, and the like to the image data based on the final radiograph, and outputs the image data to the image management apparatus 4 (Step S10). The examination information includes, for example, an examination ID, an examination date, an imaging site, and an imaging direction. The final radiograph may include, for example, predetermined image adjustment. By such a series of flows, the imaging control processing of the radiograph of the patient is completed. When the imaging of the radiograph is completed, the patient exits from the imaging room under the guidance of the user.

According to the present embodiment, the imaging support information 9a indicating that the imaging direction of the order information is different from the normal imaging direction is displayed on the imaging screen 80 before the start of positioning of the patient. This enables the user to confirm, before the start of positioning of the patient, that the imaging direction different from the normal direction is input as the order information. As a result, it is possible to reliably prevent the imaging direction from being erroneously set at the time of positioning, and it is possible to achieve a reduction in working time as compared with a case where the positioning is performed again. Furthermore, the burden on the patient can be reduced as compared with a case where repositioning is performed.

In addition, in the conventional technique, when the imaging direction of the order information is different from the normal imaging direction, the user may mistake the imaging direction for the normal imaging direction. In a case where radiographing is performed in incorrect positioning, re-imaging is required at the correct positioning according to the imaging direction of the order information. In this case, the amount of radiation exposure to the patient increases. In contrast, according to the present embodiment, even in a case where the imaging direction of the order information is different from the normal imaging direction, the user can confirm the imaging direction with low frequency using the imaging support information 9a. This makes it possible to reduce the number of times of re-imaging and reduce the amount of radiation exposure to the patient by performing radiographing in correct positioning.

### Other Embodiment 1

Next, a case where the imaging site of the input order information is an imaging site with very low frequency will be described. Note that differences from the above-described embodiment will be mainly described, and description of points common to the above-described embodiment will be omitted. Furthermore, in the description of Other Embodiment 1, the same parts as those in the above-described embodiment will be described with the same reference symbols.

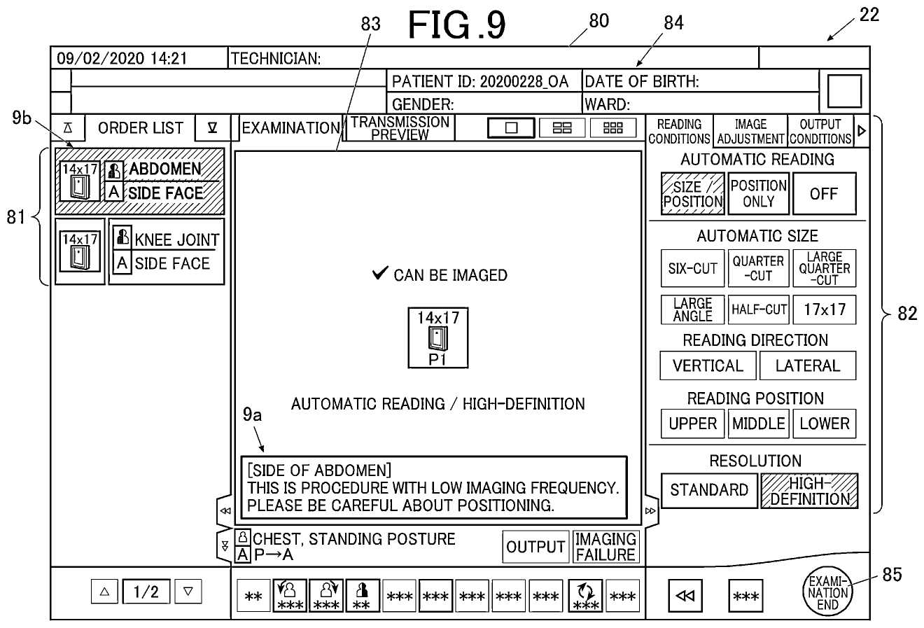

FIG. 9 illustrates an example of a configuration of an imaging screen 80 displayed on a display part 22 when the imaging site included in the order information is an imaging site with very low frequency. When the imaging site included in the order information is an imaging site with very low frequency, the controller 20 causes an imaging screen 80 to display the imaging support information 9a. To be specific, when order information 81a in which the imaging site with low frequency is the “side of abdomen” is input, the following imaging support information 9a is displayed on the imaging screen 80. The imaging support information 9a includes, for example, a text “The imaging of [side of abdomen] is a procedure with low imaging frequency. Please be careful about the positioning.”. In addition, as the imaging support information 9a, the color, font, size, brightness, and the like of the characters of the “side of abdomen” of the order information 81a selected by the imaging selector 81 may be displayed differently from other order information. That is, the characters of “side of abdomen” of the selected order information 81a may be highlighted so as to be more conspicuous than the characters of the other order information.

It may be determined as follows whether the site corresponds to the imaging site with very low frequency. The controller 20 acquires the imaging ratio of each imaging site on the basis of the imaging site of the radiograph imaged in the past. The imaging ratio of each imaging site may be set for each facility, each radiologist, and each request department. Next, the controller 20 acquires the calculated imaging ratio of the imaging site of the order information and determines whether the acquired imaging ratio of the imaging site is equal to or less than a preset threshold. Next, the controller 20 determines the imaging site having the imaging ratio being equal to or less than the threshold as an imaging site with low frequency, and causes the imaging screen 80 to display the above-described imaging support information 9a. Note that a learned model that can estimate an imaging site with low frequency obtained by machine learning may be used to determine whether the frequency of the imaging site of the order information is low.

Next, a case where imaging support information 9c is displayed in an examination list 100 will be described. When the imaging site of the order information is an imaging site with low frequency, the controller 20 causes the imaging support information 9c to be displayed not only on the imaging screen 80 but also in the examination list 100.

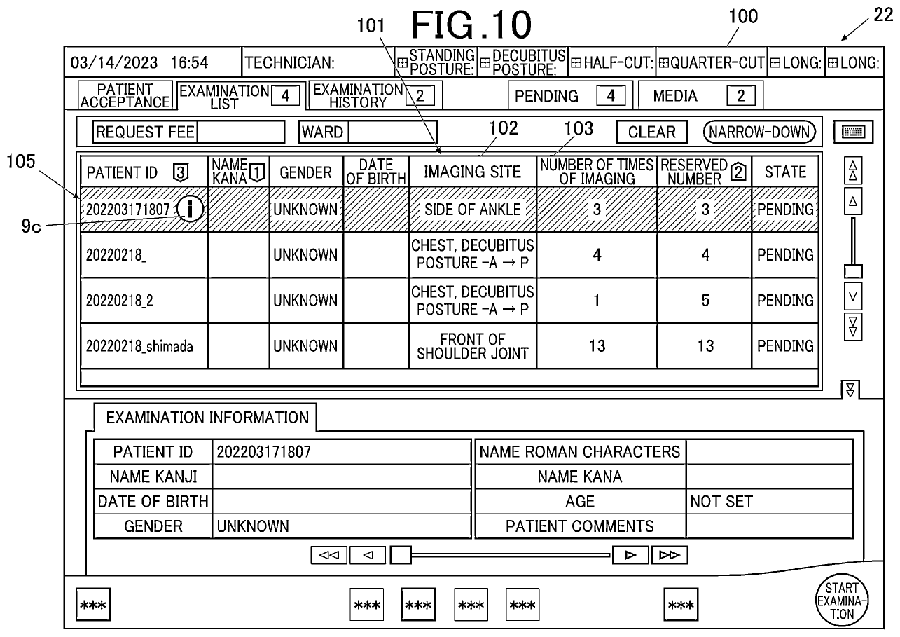

FIG. 10 illustrates an example of the imaging support information 9c displayed in the examination list 100 of the display part 22. In the examination list 100, a list of imaging order information on which examination is pending is displayed. In the examination list 100, at least patient information 101 including a patient ID and the like, imaging site information 102, and imaging number information 103 are displayed in association with each other. In the present embodiment, in the examination list 100, an imaging site with very low frequency is set as the order information 105.

The controller 20 causes the imaging support information 9c to be displayed at a position adjacent to the patient ID of the order information 105 including the imaging site with very low frequency. The imaging support information 9c is, for example, an icon indicating a caution mark. Note that the imaging support information 9c may be characters or the like in addition to icons. The order information 105 may be highlighted relative to other order information by changing the color, size, or brightness of the icon, characters, and the like. Furthermore, the imaging support information 9c may be output from the speaker by voice or sound effects indicating that the imaging site of the order information 105 has very low frequency.

### Other Embodiment 2

Next, a technician in charge of imaging a radiograph and a technician in charge of image adjustment of the imaged radiograph may work using the same imaging control apparatus 2. Such imaging is also called double focus imaging.

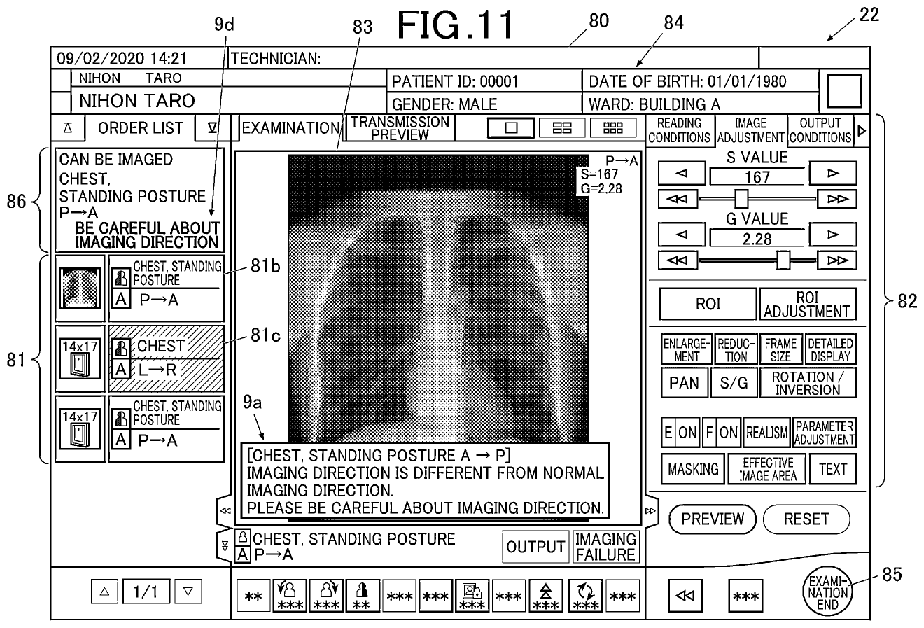

FIG. 11 illustrates an example of an imaging screen 80 displayed on a display part 22 at the time of double focus imaging. For example, three pieces of imaging order information of the same patient are displayed in an imaging selector 81 of the imaging screen 80. Of these pieces of order information, order information 81b in which the imaging site is “chest, standing posture” and the imaging direction is “AP” indicates that radiographing has been completed. The imaged radiograph is displayed in the image display area 83 of the imaging screen 80. In addition, a thumbnail image of the imaged radiograph is displayed in the order information 81b of the imaging selector 81.

When an imaging direction different from the normal imaging direction is specified, as described above, the imaging support information 9a is displayed on the imaging screen 80 of the display part 22. Therefore, the technician in charge of imaging can confirm that the current imaging direction is different from the normal imaging direction. On the other hand, since the technician in charge of imaging is in charge of image adjustment, the technician may not be able to confirm the imaging support information 9a on the imaging screen 80 at the time of imaging. Then, as illustrated in FIG. 11, the controller 20 continuously causes the imaging support information 9a to be displayed on the image display area 83 even at the time of image adjustment after the imaging of the radiograph. In this case, the controller 20 may not display the imaging support information 9a after a predetermined time has elapsed since the transition to the image adjustment. As another method, the controller 20 may not display the imaging support information 9a when a click on the imaging support information 9a window or the like is accepted.

Furthermore, in the case of the double focus imaging, there is also the following problem. On the imaging screen 80, the technician in charge of imaging may have performed image adjustment of the previous order information. Therefore, even when the imaging direction of the next order information 81c is different from the normal imaging direction, the imaging support information 9a cannot be displayed on the imaging screen 80. Then, the controller 20 causes the display area 86 above the imaging selector 81 to display the imaging support information 9d indicating that attention needs to be paid to the imaging direction of the next order information 81c. The imaging support information 9d is, for example, the wording “Confirm imaging direction”.

### Other Embodiment 3

A medical imaging system 10B according to Other Embodiment 3 controls the display and non-display of imaging support information 9a by using a detection result of the movement of a patient who has entered an imaging room. In Other Embodiment 3, differences from the medical imaging system 10A of the above-described embodiment will be mainly described, and description of points common to the above-described embodiment will be omitted. Furthermore, in the description of Other Embodiment 3, the same parts as those in the above-described embodiment will be described with the same reference symbols.

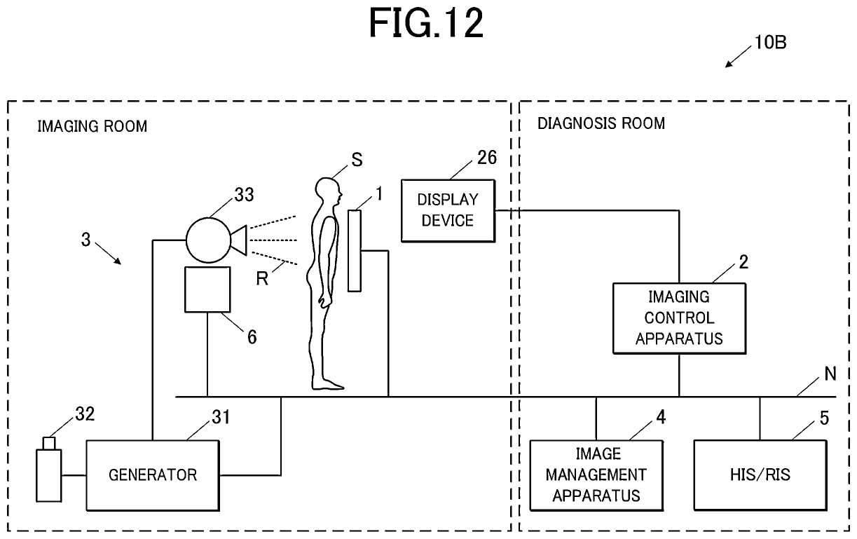

FIG. 12 is a diagram illustrating a schematic configuration of a medical imaging system 10B. The medical imaging system 10B includes a radiographic imaging apparatus 1, an imaging control apparatus 2, a radiation generation apparatus 3, an image management apparatus 4, a HIS/RIS 5, and a detector 6.

The detector 6 is arranged in the imaging room, for example, at a position adjacent to the radiation source 33 of the generation apparatus 3. The detector 6 is connected to the imaging control apparatus 2 and the like through the network N. The detector 6 detects the movement of the patient who has entered the imaging room, and supplies the detected detection signal to the imaging control apparatus 2. As the detector 6, for example, an optical camera, a human sensor, or the like is used. To be specific, the detector 6 detects a movement of the patient when the patient moves to the imaging site after entering the examination room, a movement of the patient when the patient changes the positioning, or the like. The controller 20 of the imaging control apparatus 2 acquires information on the movement of the patient on the basis of the detection result of the detector 6. This enables the controller 20 to confirm whether the current state is a state before the start of positioning or a state in which the positioning is being changed.

The controller 20 causes the imaging support information 9a to be displayed on the imaging screen 80 or the like after the completion of the previous radiographing and after the transition to the imaging screen 80 of the next radiograph. In this case, the controller 20 may display the imaging support information 9a on the imaging screen 80 or the like after confirming whether it is before the start of the positioning on the basis of the detection result of the detector 6. This enables the controller 20 to alert the user that the imaging direction of the order information is different from the normal imaging direction before the start of the positioning of the patient. Furthermore, when the controller 20 determines, from the result of detection by the detector 6, that the positioning is being changed, the controller 20 may cause the imaging support information 9a not to be displayed on the imaging screen 80 or the like. This is because the imaging support information 9a for alerting the user to the positioning is often not required after the start of positioning of the patient.

Although embodiments of the present invention have been described and illustrated in detail, the disclosed embodiments are made for purposes of illustration and example only and not limitation. The scope of the present invention should be interpreted by terms of the appended claims.

The entire disclosure of Japanese Patent Application No. 2023-111929 filed on Jul. 7, 2023 is incorporated herein by reference in its entirety.

What is claimed is:

1. A medical imaging system, comprising:
a hardware processor that acquires imaging order information including information on positioning,
determines whether the acquired imaging order information satisfies predetermined conditions, and
outputs imaging support information before start of the positioning when the imaging order information satisfies the predetermined conditions.

2. The medical imaging system according to claim 1, wherein
the imaging order information includes information on an imaging direction of an imaging site.

3. The medical imaging system according to claim 1, wherein
the imaging support information is information for an alert related to an imaging direction of an imaging site.

4. The medical imaging system according to claim 1, wherein
the hardware processor is capable of setting the predetermined conditions.

5. The medical imaging system according to claim 4, wherein
the hardware processor is capable of setting, as the predetermined conditions, information on a imaging direction and notification necessity information as to whether to provide notification of the imaging support information in association with each other.

6. The medical imaging system according to claim 2, wherein
the information on the imaging direction is an X-ray irradiation direction in a predetermined imaging direction.

7. The medical imaging system according to claim 1, further comprising:
a storage that stores information on an imaging direction and notification necessity information as to whether to provide notification of the imaging support information in association with each other, wherein
the hardware processor
specifies the notification necessity information corresponding to the information on the imaging direction stored in the storage on a basis of the information on the imaging direction acquired as the imaging order information,
determines, on the basis of the specified notification necessity information, whether the predetermined conditions are satisfied, and
outputs the imaging support information on the basis of the specified notification necessity information when the predetermined conditions are satisfied.

8. An imaging control apparatus, comprising:
a hardware processor that acquires imaging order information including information on positioning,
determines whether the acquired imaging order information satisfies predetermined conditions, and
outputs imaging support information before start of the positioning when the imaging order information satisfies the predetermined conditions.

9. The imaging control apparatus according to claim 8, wherein
the hardware processor outputs the predetermined notification to an external device.

10. The imaging control apparatus according to claim 9, wherein
the external device is an imaging apparatus.

11. The imaging control apparatus according to claim 8, wherein
the hardware processor is capable of setting the predetermined conditions, and
the hardware processor is capable of setting, as the predetermined conditions, information on a imaging direction and notification necessity information as to whether to provide notification of the imaging support information in association with each other.

12. The imaging control apparatus according to claim 8, wherein
the imaging order information includes information on an imaging direction, and
the information on the imaging direction is an irradiation direction of radiation in a predetermined imaging direction.

13. A medical imaging method causing a hardware processor to perform:
acquiring imaging order information including information on positioning;
determining whether the acquired imaging order information satisfies predetermined conditions; and
outputting imaging support information before start of the positioning when the imaging order information satisfies the predetermined conditions.

14. A non-transitory computer readable recording medium storing a program causing a computer to perform:
acquiring imaging order information including information on positioning;
determining whether the acquired imaging order information satisfies predetermined conditions; and
outputting imaging support information before start of the positioning when the imaging order information satisfies the predetermined conditions.

15. The recording medium according to claim 14, wherein
the program causes the computer to output the predetermined notification to an external device.

16. The recording medium according to claim 15, wherein
the external device is an imaging apparatus.

17. The recording medium according to claim 14, wherein
the program enables the computer to set the predetermined conditions, and
the program enables the computer to set, as the predetermined conditions, information on a imaging direction and notification necessity information as to whether to provide notification of the imaging support information in association with each other.

18. The recording medium according to claim 14, wherein
the imaging order information is information on an imaging direction, and
the information of the imaging direction is an irradiation direction of radiation in a predetermined imaging direction.
